(19) United States
(12) Patent Application Publication
(10) Pub. No.: US 2024/0043361 A1
(43) Pub. Date: Feb. 8, 2024

(54) ACETAL COMPOUND, ADDITIVE INCLUDING THE COMPOUND, AND RESIST COMPOSITION INCLUDING THE COMPOUND

(71) Applicant: MARUZEN PETROCHEMICAL CO., LTD., Tokyo (JP)

(72) Inventors: Tomohiro MASUKAWA, Ichihara-Shi (JP); Teruyo IKEDA, Ichihara-Shi (JP)

(73) Assignee: MARUZEN PETROCHEMICAL CO., LTD., Tokyo (JP)

(21) Appl. No.: 18/264,102

(22) PCT Filed: Feb. 25, 2022
(86) PCT No.: PCT/JP2022/007775

§ 371 Date: Aug. 3, 2023

(30) Foreign Application Priority Data
Feb. 26, 2021 (JP) 2021-030717

(51) Int. Cl. C07C 43/315; G03F 7/039; G03F 7/038; C07C 41/54; C07C 67/29; C07C 69/753

(52) U.S. Cl. CPC C07C 43/315; G03F 7/039; G03F 7/038; C07C 41/54; C07C 67/29; C07C 69/753; C07C 2603/74; C07C 2601/14

(57) ABSTRACT

The present invention provides: an acetal compound represented by the following general formula (1):

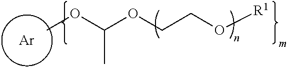

wherein R¹ represents an alkyl group having from 1 to 12 carbon atoms, a cycloalkyl group having from 5 to 12 carbon atoms or an acyl group having from 1 to 12 carbon atoms; n represents an integer from 1 to 5; m represents an integer from 2 to 4; and Ar represents a residue obtained by removing a hydrogen atom(s) from the hydroxyl groups of a polyhydric phenol having a valence of m; and a resist composition for forming a thick resist film, including the same.

### BACKGROUND OF THE INVENTION

### Field of the Invention

The present invention relates to an acetal compound, and particularly to an acetal compound useful in a resist composition. Further, the present invention relates to an additive which is used in a resist composition, and which contains the acetal compound. Still further, the present invention relates to a resist composition containing the acetal compound, and particularly to a resist composition for forming a thick resist film whose solubility in a developer changes by the action of an acid.

### Background Art

Further refinement of pattern rules is in rapid progress as LSIs tend toward higher integration and higher speed. Currently, lithography techniques using KrF excimer laser light (wavelength: 248 nm) or ArF excimer laser light (wavelength: 193 nm) are used in mass production. Further, researches and developments on lithography techniques using F2 excimer laser light (wavelength: 157 nm) having an even shorter wavelength, EUV (extreme ultraviolet) or X-rays having a wavelength shorter than these excimer lasers, or electron beams, are also in progress.

In the field of memory ICs, on the other hand, a three-dimensional memory cell structure, such as 3D-NAND, is becoming mainstream in order to achieve a higher memory capacity. Since it is necessary to increase the number of steps to be processed in the vertical direction for producing a three-dimensional memory cell structure, an increase in the film thickness of a resist film is demanded. For example, Patent Document 1 discloses a chemically amplified positive-tone photoresist composition for forming a thick resist film, which composition is used for forming a thick photoresist layer having a film thickness of from 5 to 150 μm.

### CITATION LIST

### Patent Document

Patent Document 1: JP 2008-191218 A

### SUMMARY OF THE INVENTION

### Technical Problem

However, since a resin needs to be coated in a thick layer in the case of forming a thick resist film, there is a problem that cracks are more likely to occur in the resist film after baking. There is also a problem that minute air bubbles are formed within the resist film due to the decomposition of an acid generator, in the process of etching the resist film, resulting in the occurrence of irregularities derived from the air bubbles on the pattern surface upon etching.

Accordingly, an object of the present invention is to provide a compound useful in a resist composition which allows for reducing the occurrence of cracks in the resulting thick resist film, and which is capable of forming a resist pattern with a smooth and good surface condition. Another object of the present invention is to provide a resist composition containing the compound.

### Solution to Problem

The present inventors have found out, as a result of intensive studies to achieve the above-mentioned objects, that it is possible to reduce the occurrence of cracks even in a thick resist film and to form a resist pattern with a smooth and good surface condition, by incorporating an acetal compound which is a reaction product of a polyhydric phenol and a vinyl ether having an oxyethylene chain, into a resist composition, thereby completing the present invention.

Specifically, the present invention provides the following inventions.

[1] An acetal compound represented by the following general formula (1):

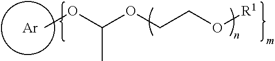

wherein, in the general formula (1), R¹ represents an alkyl group having from 1 to 12 carbon atoms, a cycloalkyl group having from 5 to 12 carbon atoms, or an acyl group having from 1 to 12 carbon atoms; n represents an integer from 1 to 5; m represents an integer from 2 to 4; and Ar represents a residue obtained by removing a hydrogen atom(s) from the hydroxyl groups of a polyhydric phenol having a valence of m.

[2] The acetal compound according to [1], wherein the vinyl ether having an oxyethylene chain is represented by the following general formula (2):

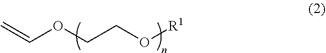

wherein, in the general formula (2), the definitions of R¹ and n are the same as those of R 1 and n in the general formula (1).

[3] The acetal compound according to [1] or [2], wherein the polyhydric phenol is a phenol having from 10 to 30 carbon atoms.

[4] The acetal compound according to [1] or [2], wherein the polyhydric phenol is a bisphenol type compound.

[5] The acetal compound according to [1] or [2], wherein the polyhydric phenol is a naphthalenediol.

[6] The acetal compound according to [1] or [2], wherein the polyhydric phenol is at least one compound selected from the group consisting of bisphenol A, 1,1,1-tris(4-hydroxyphenyl)ethane and 1,5-dihydroxynaphthalene.

[7] The acetal compound according to any one of [1] to [6], wherein the acetal compound is used in a resist composition.

[8] An additive containing the acetal compound according to any one of [1] to [7], wherein the additive is used in a resist composition.

[9] A resist composition containing:
a polymer whose solubility in a developer changes by the action of an acid;
an acid generator;
a solvent; and
the acetal compound according to any one of [1] to [7].

[10] A method for producing the acetal compound according to any one of [1] to [7],
wherein the method includes allowing a polyhydric phenol having a valence of from 2 to 4 to react with a vinyl ether represented by the following general formula (2):

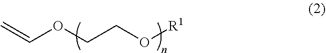

wherein, in the general formula (2), the definitions of R 1 and n are the same as those of R 1 and n in the general formula (1), in the presence of an acid.

### Advantageous Effects of Invention

According to the present invention, it is possible to prevent the occurrence of cracks in a thick resist film, and to form a resist pattern with a smooth and good pattern surface.

### BRIEF DESCRIPTION OF THE DRAWINGS

FIG. 1 shows the ¹H-NMR spectrum of Compound A synthesized in Example 1.

FIG. 2 shows the ¹³C-NMR spectrum of Compound A synthesized in Example 1.

FIG. 3 shows the ¹H-NMR spectrum of Compound B synthesized in Example 2.

FIG. 4 shows the ¹³C-NMR spectrum of Compound B synthesized in Example 2.

FIG. 5 shows the ¹H-NMR spectrum of Compound C synthesized in Example 3.

FIG. 6 shows the ¹³C-NMR spectrum of Compound C synthesized in Example 3.

FIG. 7 shows the ¹H-NMR spectrum of Compound D synthesized in Example 4.

FIG. 8 shows the ¹³C-NMR spectrum of Compound D synthesized in Example 4.

FIG. 9 shows the ¹H-NMR spectrum of Compound E synthesized in Example 5.

FIG. 10 shows the ¹³C-NMR spectrum of Compound E synthesized in Example 5.

FIG. 11 shows the ¹H-NMR spectrum of Compound F synthesized in Comparative Example 1.

FIG. 12 shows the ¹³C-NMR spectrum of Compound F synthesized in Comparative Example 1.

FIG. 13 shows the ¹H-NMR spectrum of Compound G synthesized in Comparative Example 2.

FIG. 14 shows the ¹³C-NMR spectrum of Compound G synthesized in Comparative Example 2.

### DETAILED DESCRIPTION OF THE INVENTION

### [Acetal Compound]

The acetal compound according to the present invention is a reaction product of a polyhydric phenol and a vinyl ether having an oxyethylene chain. Such an acetal compound can be used as an additive useful in a resist composition which allows for reducing the occurrence of cracks in the resulting thick resist film, and which is capable of forming a resist pattern with a smooth and good surface condition.

The acetal compound according to the present invention is represented by the following general formula (1).

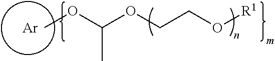

In general formula (1), R 1 represents an alkyl group having from 1 to 12 carbon atoms, a cycloalkyl group having from 5 to 12 carbon atoms, or an acyl group having from 1 to 12 carbon atoms, preferably represents an alkyl group having from 1 to 6 carbon atoms, a cycloalkyl group having from 5 to 10 carbon atoms, or an acyl group having from 1 to 12 carbon atoms, and still more preferably represents an alkyl group having from 1 to 4 carbon atoms or an acyl group having from 1 to 12 carbon atoms. n represents an integer from 1 to 5, preferably an integer from 1 to 3, and still more preferably 1 or 2. m represents an integer from 2 to 4. Ar represents a residue obtained by removing a hydrogen atom(s) from the hydroxyl groups of a polyhydric phenol having a valence of m.

The acetal compound according to the present invention can be obtained by the acetalization reaction of a polyhydric phenol with a vinyl ether having an oxyethylene chain in the presence of an acid catalyst. In the reaction of a polyhydric phenol with a vinyl ether having an oxyethylene chain, it is preferred to allow the vinyl ether in an equivalent corresponding to the valence of the phenol to react with the phenol, so that all phenolic hydroxyl groups are converted to acetal groups.

The polyhydric phenol is a phenol having a plurality of phenolic hydroxyl groups. The term “phenol” in the present specification refers in general to a compound in which a hydroxyl group(s) is/are bound to an aromatic ring. The polyhydric phenol is a dihydric to tetrahydric phenol, preferably a dihydric or trihydric phenol, and more preferably a dihydric phenol. Further, the polyhydric phenol is preferably a phenol having from 10 to 30 carbon atoms, more preferably a phenol having from 10 to 26 carbon atoms, and still more preferably a phenol having from 12 to 20 carbon atoms.

Specific examples of the polyhydric phenol include various types of bisphenols, naphthalenediols, anthracenediols, pyrenediols, 1,1,1-tris(4-hydroxyphenyl)ethane, 1,1,2,2-tetrakis(4-hydroxyphenyl)ethane and biphenylenediols. Among these, it is preferred to use at least one compound selected from the group consisting of bisphenol A, 1,1,1-tris(4-hydroxyphenyl)ethane and 1,5-dihydroxynaphthalene.

The vinyl ether having an oxyethylene chain is preferably a compound represented by the formula (2).

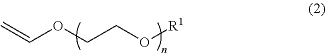

The definitions of R¹ and n in the general formula (2) are the same as those of R¹ and n in the general formula (1).

Examples of compounds represented by the general formula (2) include the following.

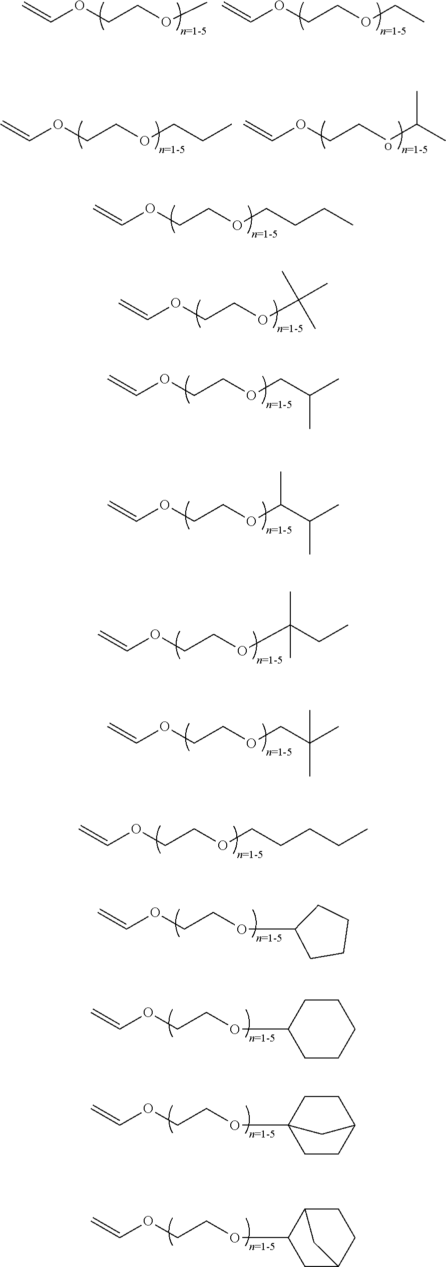

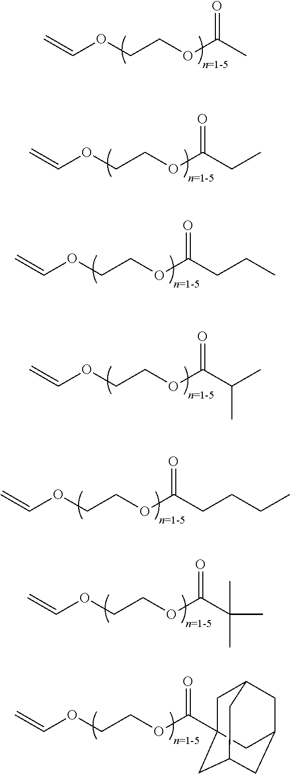

* n in each formula represents the number of repetitions of the oxyethylene chain.

Examples of the acid catalyst to be used in the acetalization reaction include: inorganic acids such as sulfuric acid, nitric acid, hydrochloric acid and phosphoric acid; carboxylic acids such as formic acid, acetic acid, butyric acid and trifluoroacetic acid; sulfonic acids such as methanesulfonic acid, benzenesulfonic acid and toluenesulfonic acid; and phosphonic acids such as methanephosphonic acid and benzenephosphonic acid. Among these, a carboxylic acid or a sulfonic acid is preferred from the viewpoint of inhibiting the polymerization reaction of the vinyl ether

The amount of the acid catalyst to be used cannot be defined in general, since it varies depending on the type of the acid used. However, the amount of the acid catalyst to be used is usually from 1 to 5,000 ppm and preferably from 1 to 2,000 ppm, with respect to the entire reaction system. When the amount of the acid catalyst to be used is within the range described above, side reactions, such as the polymerization reaction of the vinyl ether, are less likely to occur, and a sufficient reaction velocity is more likely to be obtained.

The solvent to be used in the acetalization reaction may be any solvent capable of stably dissolving a polymer having phenolic hydroxyl groups, a vinyl ether and an acid catalyst, which are raw materials, as well as a product obtained by the acetalization reaction. Specific examples of the solvent include: esters such as methyl acetate, ethyl acetate, isopropyl acetate, propyl acetate, butyl acetate, methyl propionate, methyl lactate, and ethyl lactate; glycol ether esters such as ethylene glycol monomethyl ether acetate, ethylene glycol monoethyl ether acetate, propylene glycol monomethyl ether acetate and propylene glycol monoethyl ether acetate; ethers such as tetrahydrofuran, 1,4-dioxane and ethylene glycol dimethyl ether; and aromatic hydrocarbons such as toluene and xylene. These solvents can be used singly, or in combination of two or more kinds thereof.

The acetalization reaction is carried out preferably at a temperature of from 25 to 120° C., more preferably from 30 to 100° C., and still more preferably from 30 to 80° C.

After the acetalization reaction, it is preferred to add a basic compound to the resulting reaction solution to neutralize the acid catalyst, or to remove the acid catalyst with an anion exchange resin. Specific examples of the basic compound include: hydroxides of alkali metals such as sodium and potassium; alkali metal compounds such as carbonates and bicarbonates; ammonia water and ammonia gas; amines such as trimethylamine and triethylamine; pyridines such as pyridine and methylpyridine; and quaternary ammonium compounds such as tetraalkylammonium hydroxide. The neutralization of the acid catalyst with an amine or a quaternary ammonium compound, or the removal of the acid catalyst with an anion exchange resin is preferred.

### <Resist Composition>

The resist composition according to the present invention contains at least: a polymer whose solubility in a developer changes by the action of an acid; an acid generator; a solvent; and the acetal compound according to the present invention; and may further contain another additive such as an acid diffusion inhibitor. In particular, the acetal compound according to the present invention can be suitably used in a resist composition for forming a thick resist film, which is used for forming a thick resist film having a film thickness of 1 μm or more, preferably a film thickness of 1 μm or more and 100 μm or less.

In the case of a resist composition for forming a thick resist film, the content of the acetal compound according to the present invention in the composition is preferably from 1 to 50% by mass, more preferably from 1 to 30% by mass, still more preferably from 1 to 20% by mass, and particularly preferably from 1 to 15% by mass. Further, the content of the polymer is preferably from 10 to 80% by mass, more preferably from 20 to 60% by mass, and still more preferably from 25 to 50% by mass.

The polymer to be used in the resist composition according to the present invention is a polymer whose solubility in a developer changes by the action of an acid, and can be selected arbitrarily from those commonly used in chemically amplified resist applications.

In particular, a polymer containing a unit derived from hydroxystyrene or a repeating unit having a structure in which the hydroxyl group of hydroxystyrene is protected by a group (hereinafter, referred to as “acid-dissociable group”) capable of being eliminated by the action of an acid is preferred.

Further, the polymer may also contain a repeating unit having a structure in which the carboxyl group of acrylic acid or methacrylic acid is protected by an acid-dissociable group.

Acid-dissociable groups having various structures are known, and the acid-dissociable group to be used is not particularly limited. Specific examples of the acid-dissociable group include: saturated hydrocarbon groups such as tert-butyl group, tert-amyl group, 1-methyl-1-cyclopentyl group, 1-ethyl-1-cyclopentyl group, 1-methyl-1-cyclohexyl group, 1-ethyl-1-cyclohexyl group, 2-methyl-2-adamantyl group, 2-ethyl-2-adamantyl group, 2-propyl-2-adamantyl group, 2-(1-adamantyl)-2-propyl group, 8-methyl-8-tricyclo[5.2.1.0^{2,6}]decanyl group, 8-ethyl-8-tricyclo[5.2.1.0^{2,6}]decanyl group, 8-methyl-8-tetracyclo[4.4.0.1^{2,5}.1^{7,10}]dodecanyl group and 8-ethyl-8-tetracyclo[4.4.0.1²⁵.1^{7,10}]dodecanyl group; and oxygen-containing hydrocarbon groups such as 1-methoxyethyl group, 1-ethoxyethyl group, 1-iso-propoxyethyl group, 1-n-butoxyethyl group, 1-tert-butoxyethyl group, 1-cyclopentyloxyethyl group, 1-cyclohexyloxyethyl group, 1-tricyclo[5.2.1.0^{2,6}]decanyloxyethyl group, methoxymethyl group, ethoxymethyl group, iso-propoxymethyl group, n-butoxymethyl group, tert-butoxymethyl group, cyclopentyloxymethyl group, cyclohexyloxymethyl group, tricyclo[5.2.1.0^{2,6}]decanyloxymethyl group, tetrahydropyranyl group and tert-butoxycarbonyl group.

The polymer may contain, in addition to the repeating units described above, a repeating unit containing a polar group such as an alcoholic hydroxyl group, a lactone, or a sultone, for the purpose of improving the adhesion of the polymer to a substrate. One example of a monomer which gives such a repeating unit may be, for example, 2-hydroxyethyl (meth)acrylate, 3-hydroxy-1-adamantyl methacrylate, γ-butyrolactone-α-(meth)acrylate, norbornane lactone (meth)acrylate, 2-methacryloyloxyacetoxy-4,5-oxathiatricyclo[4.2.1.03,7]nonane=5,5-dioxide, or the like.

Further, the polymer may contain another repeating unit which does not contain an acid-dissociable group. One example of a monomer which gives such a repeating unit may be, for example, styrene, 2-vinylnaphthalene, methyl (meth)acrylate, 1-adamantyl (meth)acrylate, or the like.

The acid generator to be used can be selected as appropriate from compounds which have hitherto been proposed as acid generators for use in chemically amplified resists. Examples of such compounds include: onium salts such as iodonium salts and sulfonium salts; oxime sulfonates; diazomethanes such as bisalkyl sulfonyldiazomethanes and bisaryl sulfonyldiazomethanes; nitrobenzyl sulfonates; iminosulfonates; and disulfones. Among these, onium salts are preferred. These acid generators maybe used singly, or in combination of two or more kinds thereof.

The acid diffusion inhibitor can be selected as appropriate from compounds which have hitherto been proposed as acid diffusion inhibitors for use in chemically amplified resists. Examples of such compounds include nitrogen-containing organic compounds, and primary to tertiary alkylamines and hydroxyalkylamines are preferred. In particular, tertiary alkylamines and tertiary hydroxyalkylamines are preferred. Among these, triethanolamine and triisopropanolamine are particularly preferred. These acid diffusion inhibitors may be used singly, or in combination of two or more kinds thereof.

The solvent may be any solvent capable of dissolving the respective components constituting the resist composition to form a homogeneous solution. For example, it is possible to use a single solvent or a mixed solvent of two or more kinds of solvents, arbitrarily selected from those known as solvents for forming coating films. Solvents having at least one or more polar groups selected from the group consisting of a ketone bond, an ester bond, an ether bond and a hydroxy group are preferred, because such solvents have an excellent solubility. Among these, solvents having a boiling point at normal pressure of from 110 to 220° C. are particularly preferred, because such solvents have a moderate evaporation rate in baking after spin coating, and have excellent film forming properties. Specific examples of such solvents include: solvents having a ketone bond, such as methyl isobutyl ketone, methyl isoamyl ketone, methyl amyl ketone, and cyclohexanone; solvents having an ether bond and a hydroxy group, such as propylene glycol monomethyl ether (PGME) and propylene glycol monoethyl ether; solvents having an ether bond and an ester bond, such as propylene glycol monomethyl ether acetate (PGMEA), propylene glycol monoethyl ether acetate and ethyl 3-ethoxypropionate; solvents having an ester bond and a hydroxy group, such as methyl lactate and ethyl lactate; and solvents having an ester bond, such as γ-butyrolactone. Among these, PGMEA, PGME, γ-butyrolactone and ethyl lactate are preferred.

Optionally, the resist composition can further contain, as appropriate and as necessary, a compound(s) commonly used as resist additives, such as, for example: an organic carboxylic acid or an oxoacid of phosphorus intended for preventing the deterioration in sensitivity of the acid generator, or improving the shape, the post exposure stability and the like of the resulting resist pattern; an additional resin for improving the performance of the resulting resist film; a surfactant for improving coating properties; a dissolution inhibitor; a plasticizer; a stabilizer; a colorant; an antihalation agent; and/or a dye.

### EXAMPLES

Embodiments of the present invention will now be described in detail with reference to Examples. It is noted, however, that the present invention is in no way limited to these Examples. In the following Examples, the term “part(s)” is based on mass unless otherwise specified.

Analyses in the present Examples are carried out as follows.

### [Identification of Compound]

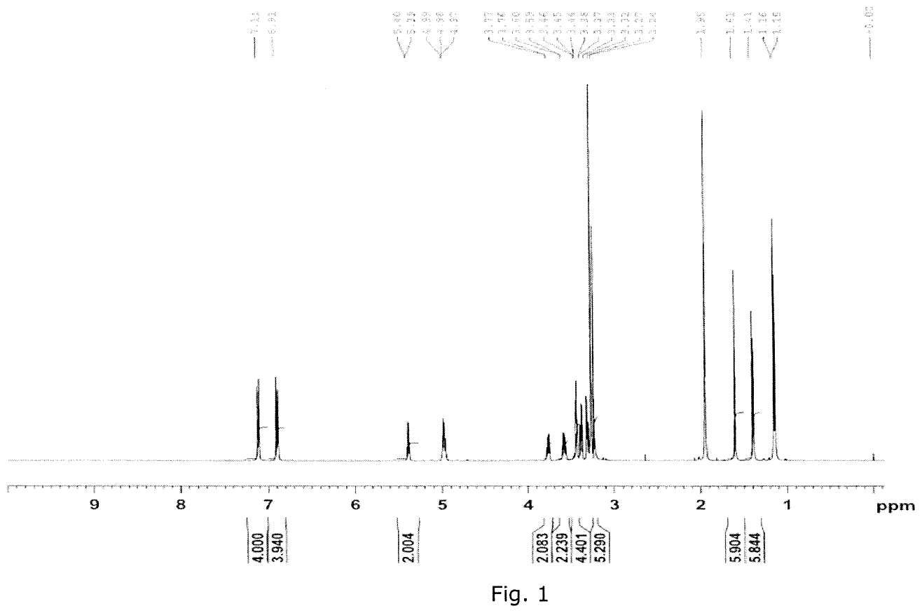

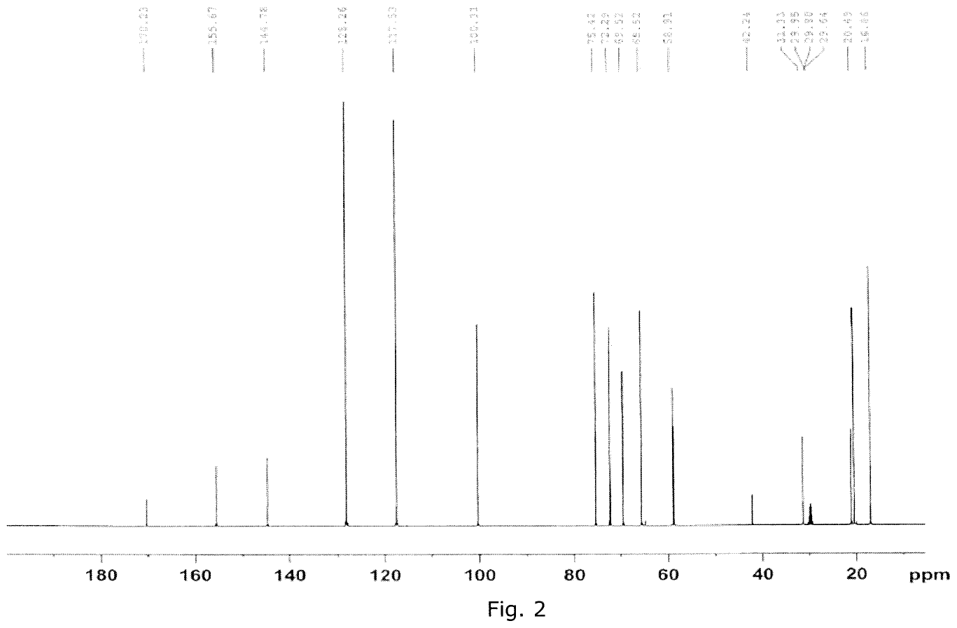

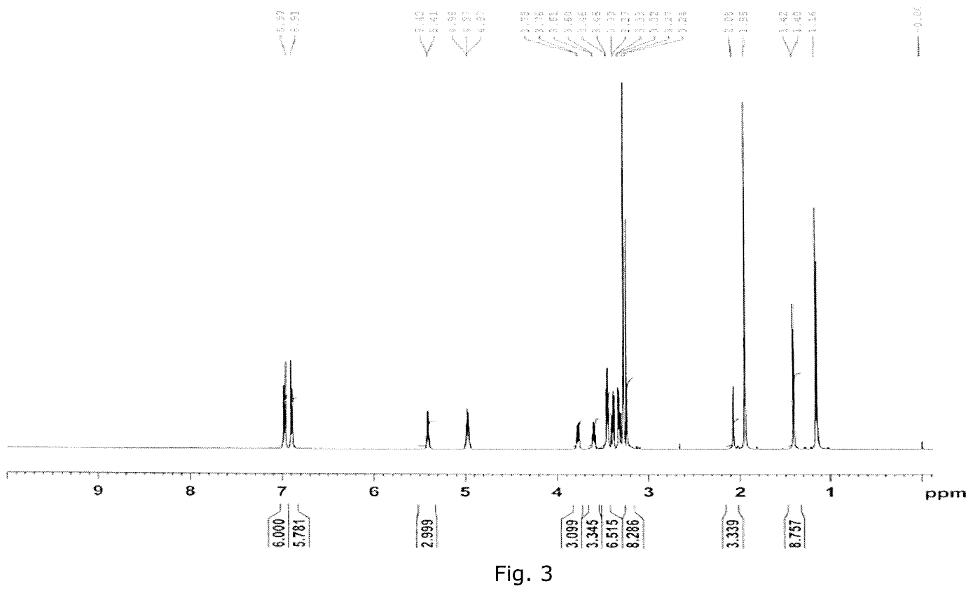

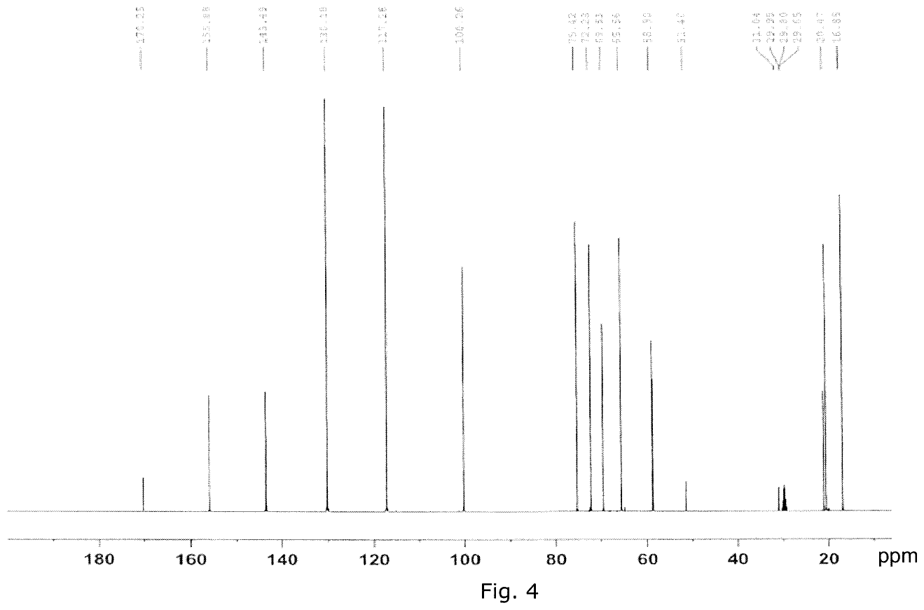

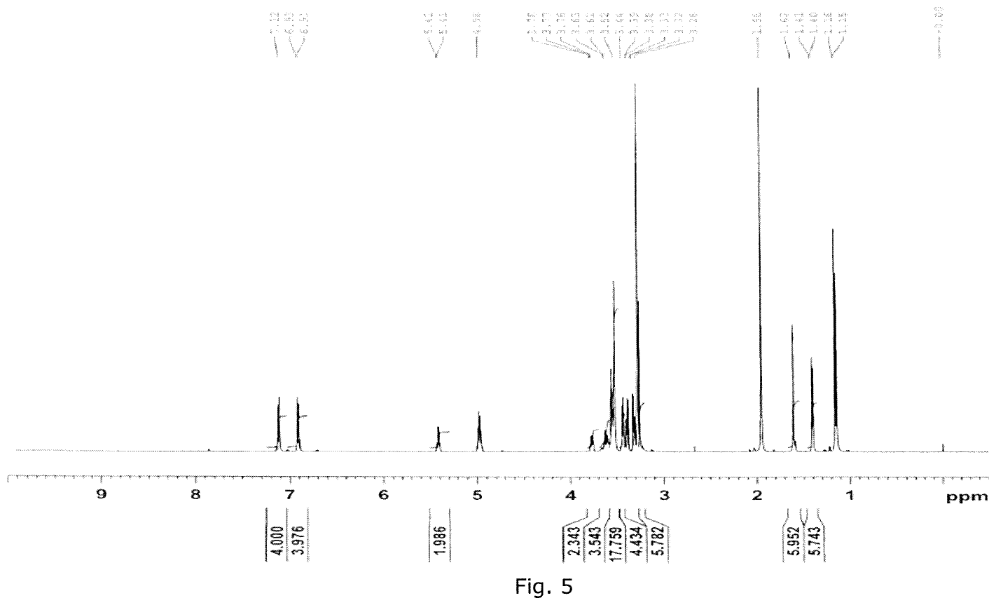

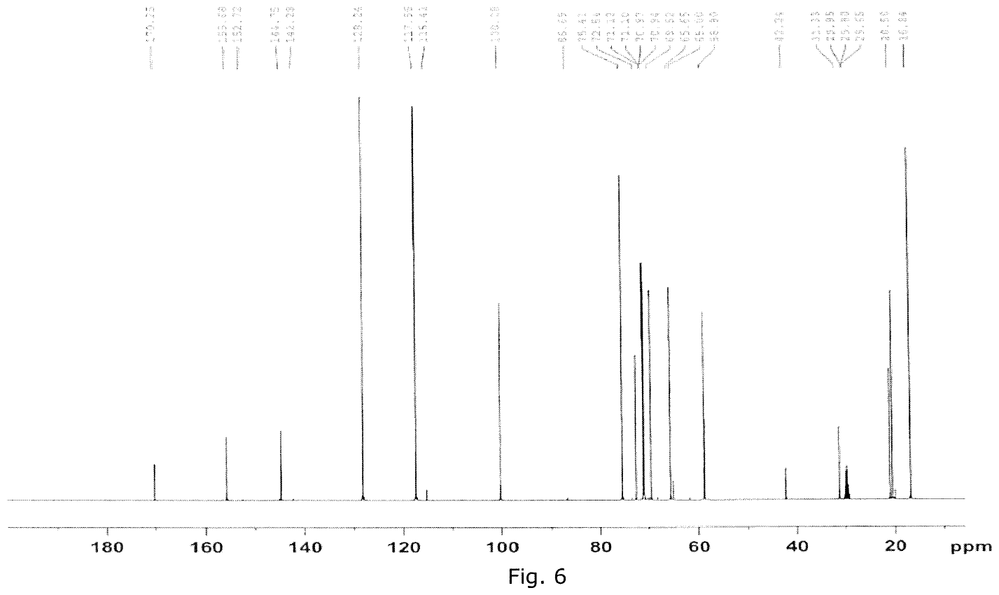

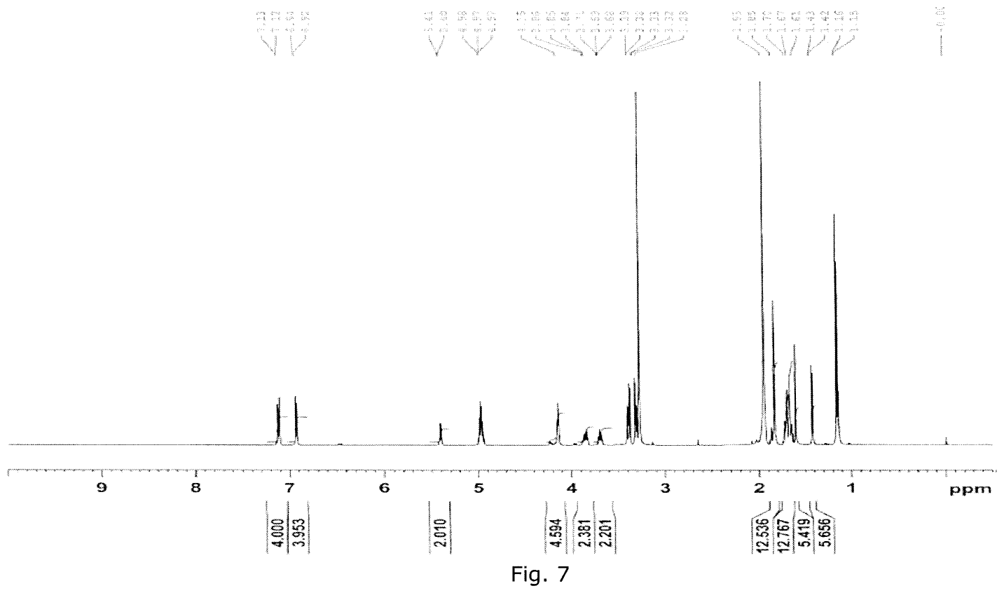

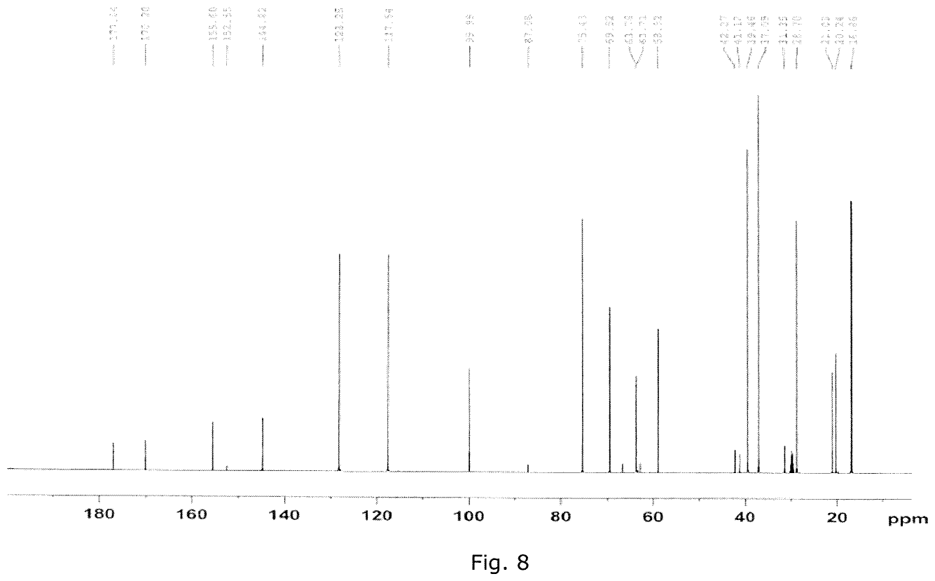

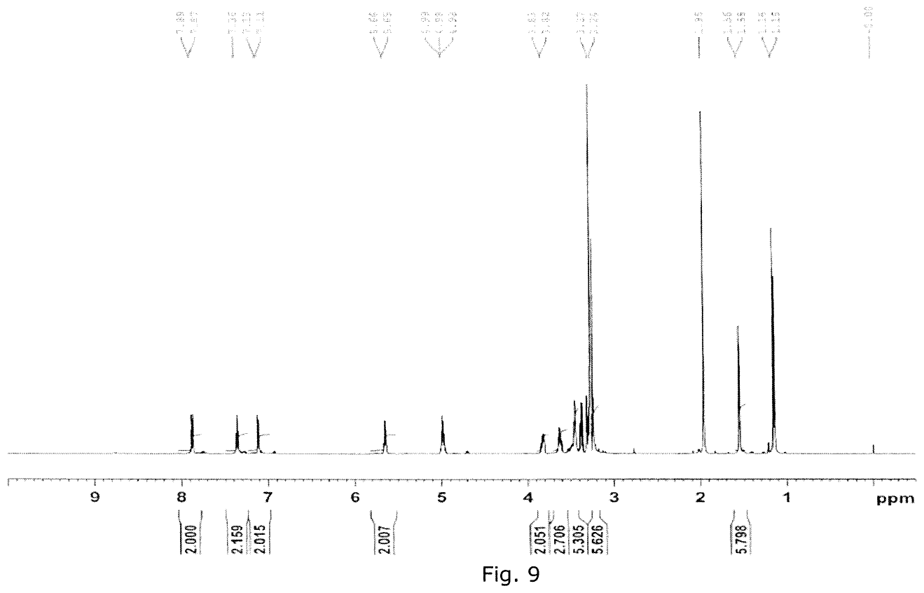

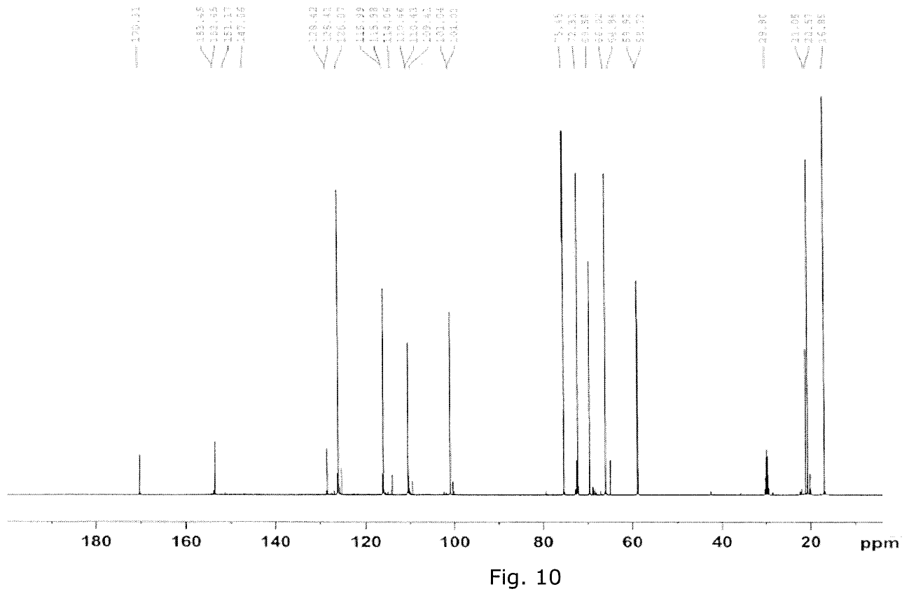

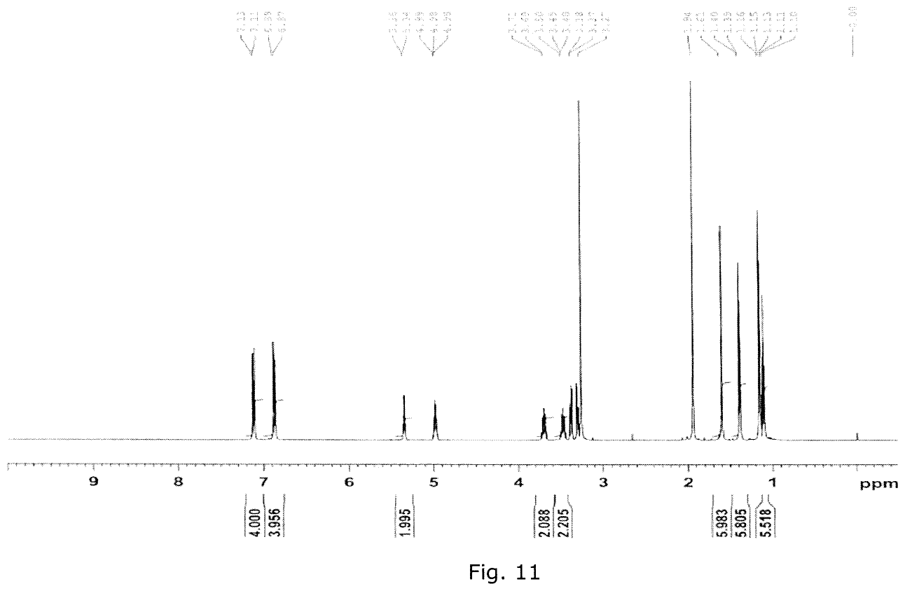

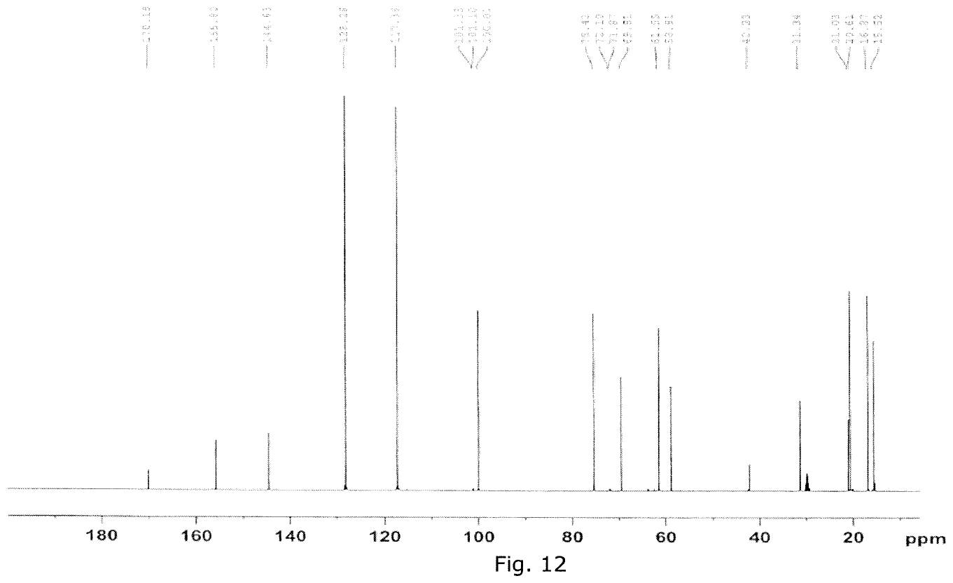

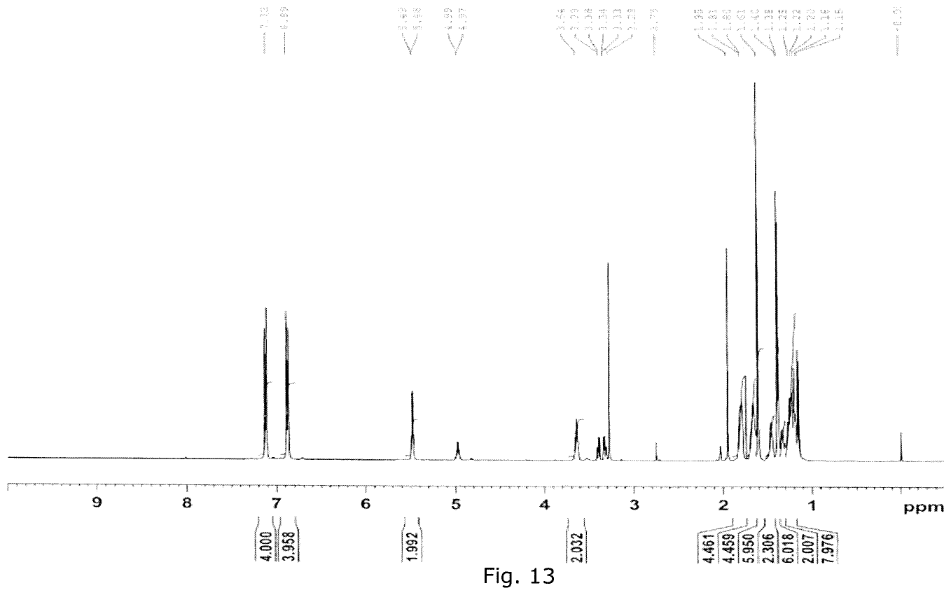

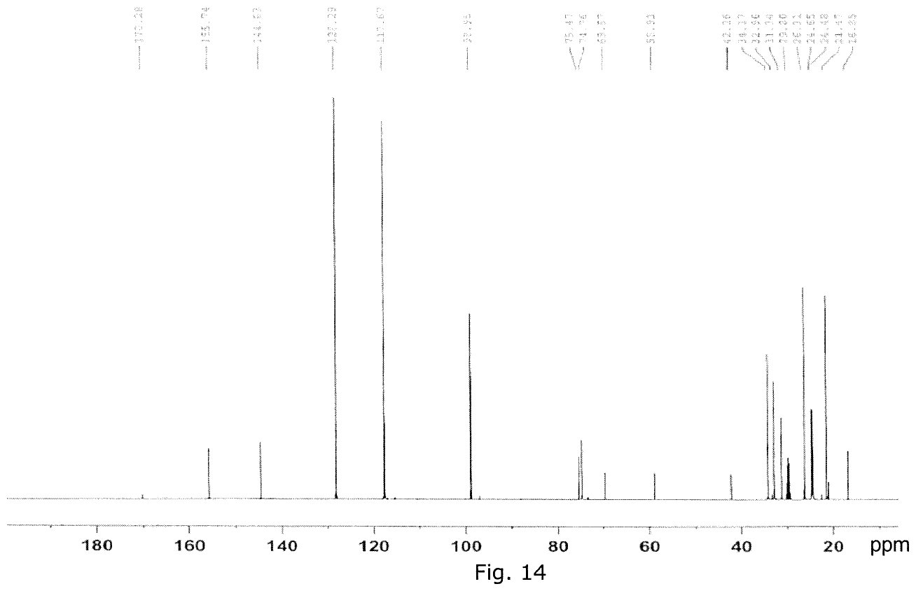

The identification of each compound synthesized as will be described below was carried out by ¹H-NMR and ¹³C-NMR. The ¹H-NMR spectra and the ¹³C-NMR spectra of the respective compounds are shown in FIG. 1 to FIG. 14. It is noted that, since each synthesized compound was analyzed in the state of a solution containing a solvent, without isolating the compound, the peaks of propylene glycol monomethyl ether acetate as the solvent are present in a mixed state along with those of the compound, in each NMR spectrum. The peaks of propylene glycol monomethyl ether acetate in ¹H-NMR were ppm=1.16 (d), 1.95, 3.27, 3,32 to 3.38 (dd), and 4.98 (m), and the peaks of propylene glycol monomethyl ether acetate in ¹³C-NMR spectra were ppm=16.86, 21.03, 58.91, 69.52, 75.42, and 170.23.

Apparatus: AVANCE, manufactured by Bruker Corporation

Frequency: 500 MHz

Deuterated solvent: acetone-d6

Internal standard: TMS

Measurement temperature: 30° C.

### [Weight Average Molecular Weight and Degree of Dispersion of Polymer]

The weight average molecular weight (Mw) and the degree of dispersion (Mw/Mn) of the polymer synthesized as will be described below were measured by GPC (gel permeation chromatography), using polystyrene as a standard.

Measuring apparatus: HPLC-8220 GPC, manufactured by Tosoh Corporation

Detector: differential refractive index (RI) detector

Columns: Shodex GPC KF804×three columns (manufactured by Showa Denko K.K.)

Eluent: tetrahydrofuran

Flow rate: 1.0 mL/min

Temperature: 40° C.

Calibration curve: prepared using a polystyrene standard sample (manufactured by Tosoh Corporation)

### [Polymer Composition]

The composition ratio of the respective monomers in the synthesized polymer was analyzed by ¹³C-NMR. A quantity of 2.0 g of a solution of the polymer after adjusting the concentration and 0.1 g of Cr (III) acetylacetonate were dissolved in 1.0 g of deuterated acetone, to prepare a sample for analysis.

Apparatus: AVANCE 400, manufactured by Bruker Corporation

Nuclide: ¹³C

Measurement method: inverse gated decoupling

### [Synthesis of Resist Additive]

### Example 1

### Synthesis of Compound A

Bisphenol A and propylene glycol monomethyl ether acetate (hereinafter, referred to as “PGMEA”) were introduced into an eggplant flask, and concentrated under reduced pressure at 40° C., to prepare a solution in which the amount of water in the solution was 500 ppm or less and the concentration of Bis-A therein was 40% by mass. Into a reaction vessel equipped with a thermometer, a cooling tube, and a stirrer, 2,680.0 g of the Bis-A/PGMEA solution described above and 63.8 g of a 20% by mass trifluoroacetic acid/PGMEA solution were introduced, and the mixture was heated to 60° C. with stirring in a nitrogen gas stream. To the resulting solution, a mixed solution of 1,055.4 g of 2-methoxyethyl vinyl ether (hereinafter, referred to as “MOVE”) and 468.2 g of PGMEA was added dropwise over 60 minutes. After the completion of the dropwise addition, the reaction was allowed to continue for another 4 hours. After the completion of the reaction, the reaction solution was passed through 243.5 g of Amberlyst B20-HG·DRY which had been filled in a column over 6 hours, to remove trifluoroacetic acid as the catalyst. The reaction solution was then concentrated at 40° C. under reduced pressure, to obtain a PGMEA solution containing Compound A at a concentration of 50% by mass.

The thus obtained Compound A was confirmed to have the structure represented by the following formula (3) by NMR analyses. Further, the resulting compound had an HPLC purity of 98.7%.

### Compound A:

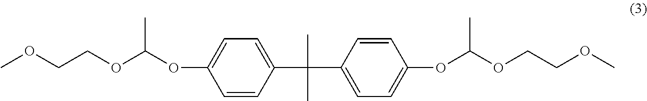

The results of the NMR analyses of Compound A are as follows.

¹H-NMR (500 MHz, acetone-d6): δ ppm=1.41 (6H), 1.61 (6H), 3.24 (6H), 3.45 (4H), 3.60 (2H), 3.77 (2H), 5.40 (2H), 6.91 (4H), 7.11 (4H)

¹³C-NMR (500 MHz, acetone-d6): δ ppm=20.49, 31.33, 42.24, 58.91, 65.52, 72.29, 100.31, 117.53, 128.26, 144.78, 155.67

### Example 2

### Synthesis of Compound B

1,1,1-Tris(4-hydroxyphenyl)ethane (hereinafter, referred to as “THPE”) and PGMEA were introduced into an eggplant flask, and concentrated under reduced pressure at 40° C., to prepare a solution in which the amount of water in the solution was 500 ppm or less and the concentration of THPE therein was 20% by mass. Into a reaction vessel equipped with a thermometer, a cooling tube, and a stirrer, 2,966.5 g of the THPE/PGMEA solution described above and 4.2 g of a 1% by mass methanesulfonic acid/PGMEA solution were introduced, and the mixture was heated to 60° C. with stirring in a nitrogen gas stream. To the resulting solution, a mixed solution of 672.5 g of 2-methoxyethyl vinyl ether (hereinafter, referred to as “MOVE”) and 552.4 g of PGMEA was added dropwise over 120 minutes. After the completion of the dropwise addition, the reaction was allowed to continue for another 2 hours. After the completion of the reaction, the reaction solution was passed through 243.5 g of Amberlyst B20-HGDRY which had been filled in a column over 6 hours, to remove methanesulfonic acid as the catalyst.

The reaction solution was then concentrated at 40° C. under reduced pressure, to obtain a PGMEA solution containing Compound B at a concentration of 50% by mass.

The thus obtained Compound B was confirmed to have the structure represented by the following formula (4) by NMR analyses. Further, the resulting compound had an HPLC purity of 94.2%.

### Compound B:

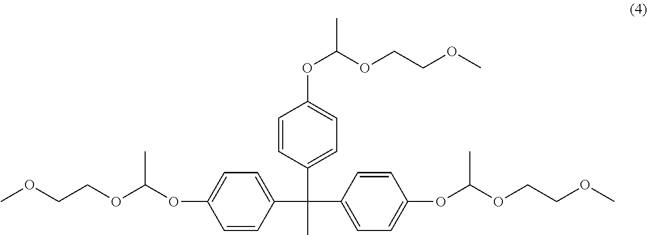

The results of the NMR analyses of Compound B are as follows.

¹H-NMR (400 MHz, acetone-d6): δ ppm=1.41 (9H), 2.08 (3H), 3.24 (9H), 3.46 (6H), 3.61 (3H), 3.77 (3H), 5.42 (3H), 6.91 (6H), 6.97 (6H)

¹³C-NMR (400 MHz, acetone-d6): δ ppm=20.47, 31.04, 51.40, 58.90, 63.56, 72.28, 100.26, 117.26, 130.18, 143.49, 155.88

### Example 3

### Synthesis of Compound C

Bisphenol A and PGMEA were introduced into an eggplant flask, and concentrated under reduced pressure at 40° C., to obtain a solution in which the amount of water in the solution was 500 ppm or less and the concentration of Bis-A therein was 40% by mass. Into a reaction vessel equipped with a thermometer, a cooling tube, and a stirrer, 200.8 g of the 40% by mass bisphenol A/PGMEA solution described above and 6.8 g of a 20% by mass trifluoroacetic acid/PGMEA solution were introduced, and the mixture was heated to 60° C. with stirring in a nitrogen gas stream. To the resulting solution, a mixed solution of 147.2 g of 2-(2-(2-methoxyethoxy)ethoxy)ethyl vinyl ether (hereinafter, referred to as “TEGVE) and 101.6 g of PGMEA was added dropwise over 60 minutes. After the completion of the dropwise addition, the reaction was allowed to continue for another 6 hours. After the completion of the reaction, the reaction solution was passed through 26.1 g of Amberlyst B20-HG·DRY which had been filled in a column over 6 hours, to remove trifluoroacetic acid as the catalyst. The reaction solution was then concentrated at 40° C. under reduced pressure, to obtain a PGMEA solution containing Compound C at a concentration of 50% by mass.

The thus obtained Compound C was confirmed to have the structure represented by the following formula (5) by NMR analyses. Further, the resulting compound had an HPLC purity of 94.9%.

### Compound C:

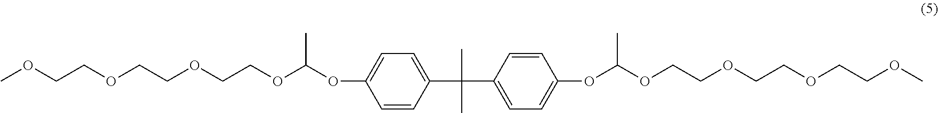

The results of the NMR analyses of Compound C are as follows.

¹H-NMR (400 MHz, acetone-d6): δ ppm=1.41 (6H), 1.62 (6H), 3.24 (6H), 3.44 to 3.78 (24H), 5.42 (2H), 6.92 (4H), 7.12 (4H)

¹³C-NMR (400 MHz, acetone-d6): δ ppm=20.50, 31.33, 42.24, 58.90, 65.65, 70.94-71.12, 72.54, 100.28, 117.56, 128.24, 144.75, 155.68

### Example 4

### Synthesis of Compound D

A quantity of 750.0 g of bisphenol A, 1768.4 g of 2-(vinyloxy)ethyl 1-adamantane carboxylate (hereinafter, referred to as “ACVE”), and PGMEA were introduced into an eggplant flask, and concentrated under reduced pressure at 40° C., to obtain a PGMEA solution in which the amount of water in the solution was 500 ppm or less and the total concentration of bisphenol A and ACVE therein was 55% by mass. Into an eggplant flask equipped with a thermometer, a cooling tube, and a stirrer, thermometer, 906.8 g of the solution described above and 112.4 g of a 20% by mass trifluoroacetic acid/PGMEA solution were introduced, and the mixture was heated to 50° C. with stirring in a nitrogen gas stream. Thereafter, the reaction was allowed to continue for 8 hours. After the completion of the reaction, the reaction solution was passed through 430.0 g of Amberlyst B20-HGDRY which had been filled in a column over 6 hours, to remove trifluoroacetic acid as the catalyst. The reaction solution was then concentrated at 40° C. under reduced pressure, to obtain a PGMEA solution containing Compound D at a concentration of 50% by mass.

The thus obtained Compound D was confirmed to have the structure represented by the following formula (6) by NMR analyses. Further, the resulting compound had an HPLC purity of 98.5%.

### Compound D:

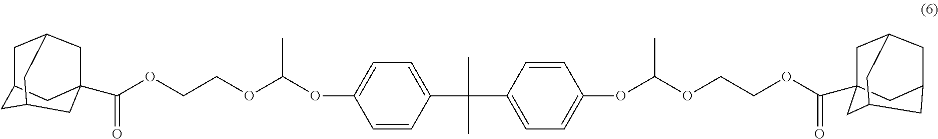 [SMILES: CC(OCCOC(=O)C12CC3CC(CC(C3)C1)C2)Oc1ccc(C(C)(C)c2ccc(OC(C)OCCOC(=O)C34CC5CC(CC(C5)C3)C4)cc2)cc1]

The results of the NMR analyses of Compound D are as follows.

¹H-NMR (400 MHz, acetone-d6): δ ppm=1.43 (6H), 1.61(6H), 1.67 to 1.70 (12H), 1.85 (12H), in the vicinity of 1,96 (6H), 3.69 (2H), 3.85 (2H), 4.14 (4H), 5.41 (2H), 6.93 (4H), 7.13 (4H)

¹³C-NMR (400 MHz, acetone-d6): δ ppm=20.24, 28.70, 31.35, 37.09, 39.46, 41.12, 42.27, 63.71, 63.78, 99.99, 117.54, 128.25, 144.82, 155.60, 177.04

### Example 5

### Synthesis of Compound E

1,5-Dihydroxynaphthalene and PGMEA were introduced into an eggplant flask, and concentrated under reduced pressure at 40° C., to prepare a solution in which the amount of water in the solution was 500 ppm or less and the concentration of 1,5-dihydroxynaphthalene therein was 40% by mass. Into an eggplant flask equipped with a thermometer, a cooling tube and a stirrer, 243.8 g of the 1,5-dihydroxynaphthalene/PGMEA solution described above and 0.5 g of a 1% by mass methanesulfonic acid/PGMEA solution were introduced, and the mixture was heated to 60° C. with stirring in a nitrogen gas stream. To the resulting solution, a mixed solution of 138.1 g of 2-methoxyethyl vinyl ether and 88.5 g of PGMEA was added dropwise over 60 minutes. After the completion of the dropwise addition, the reaction was allowed to continue for another 6 hours. After the completion of the reaction, the reaction solution was passed through 10.7 g of Amberlyst B20-HG·DRY which had been filled in a column over 6 hours, to remove methanesulfonic acid as the catalyst. The reaction solution was then concentrated at 40° C. under reduced pressure, to obtain a solution containing Compound E at a concentration of 50% by mass.

The thus obtained Compound E was confirmed to have the structure represented by the following formula (7) by NMR analyses. Further, the resulting compound E had an HPLC purity of 88.8%.

### Compound E:

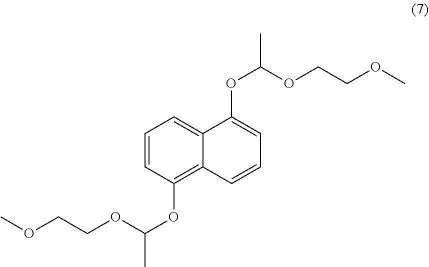

The results of the NMR analyses of Compound E are as follows.

¹H-NMR (400 MHz, acetone-d6): δ ppm=1.56 (6H), 3.24 (6H), 3.4 to 3.9 (8H), 5.66 (2H), 7.12 (2H), 7.36 (2H), 7.88 (2H)

¹³C-NMR (400 MHz, acetone-d6): δ ppm=20.57, 58.72, 66.02, 72.31, 101.04, 110.45, 115.99, 126.07, 128.42, 153.45

### Comparative Example 1

### Synthesis of Compound F

Bisphenol A and PGMEA were introduced into an eggplant flask, and concentrated under reduced pressure at 40° C., to obtain a solution in which the amount of water in the solution was 500 ppm or less and the concentration of bisphenol A therein was 40% by mass. Into an eggplant flask equipped with a thermometer, a cooling tube, and a stirrer, 337.6 g of the bisphenol A/PGMEA solution described above and 7.0 g of a 20% by mass trifluoroacetic acid/PGMEA solution were introduced, and the mixture was heated to 40° C. with stirring in a nitrogen gas stream. To the resulting solution, a mixed solution of 98.1 g of ethyl vinyl ether and 25.0 g of PGMEA was added dropwise over 60 minutes. After the completion of the dropwise addition, the reaction was allowed to continue for another 4 hours. After the completion of the reaction, the reaction solution was passed through 26.7 g of Amberlyst B20-HG·DRY which had been filled in a column over 6 hours, to remove trifluoroacetic acid as the catalyst. The reaction solution was then concentrated at 40° C. under reduced pressure, to obtain a solution containing Compound F at a concentration of 50% by mass.

The thus obtained Compound F was confirmed to have the structure represented by the following formula (8) by NMR analyses. Further, the resulting compound had an HPLC purity of 96.6%.

### Compound F:

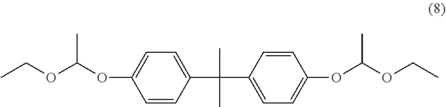 [SMILES: CCOC(C)Oc1ccc(C(C)(C)c2ccc(OC(C)OCC)cc2)cc1]

The results of the NMR analyses of Compound F are as follows.

¹H-NMR (400 MHz, acetone-d6): δ ppm=1.11 (6H), 1.40 (6H), 1.61 (6H), 3.49 (2H), 3.71 (2H), 5.35 (2H), 6.89 (4H), 7.12 (4H)

¹³C-NMR (400 MHz, acetone-d6): δ ppm=15.52, 20.61, 31.34, 42.24, 61.55, 100.01, 117.38, 128.26, 144.63, 155.80

### Comparative Example 2

### Synthesis of Compound G

Bisphenol A and PGMEA were introduced into an eggplant flask, and concentrated under reduced pressure at 40° C., to prepare a PGMEA solution in which the amount of water in the solution was 500 ppm or less and the concentration of bisphenol A therein was 40% by mass. Into an eggplant flask equipped with a thermometer, a cooling tube, and a stirrer, 2,401.9 g of the bisphenol A/PGMEA solution described above and 63.9 g of a 20% by mass trifluoroacetic acid/PGMEA solution were introduced, and the mixture was heated to 60° C. with stirring in a nitrogen gas stream. To the resulting solution, a mixed solution of 1,168.4 g of cyclohexyl vinyl ether and 637.0 g of PGMEA was added dropwise over 60 minutes. After the completion of the dropwise addition, the reaction was allowed to continue for another 6 hours. After the completion of the reaction, the reaction solution was passed through 243.7 g of Amberlyst B20-HGDRY which had been filled in a column over 6 hours, to remove trifluoroacetic acid as the catalyst. The reaction solution was then concentrated at 40° C. under reduced pressure, to obtain a solution containing Compound G at a concentration of 50% by mass.

The thus obtained Compound G was confirmed to have the structure represented by the following formula (9) by NMR analyses. Further, the resulting compound had an HPLC purity of 97.8%.

### Compound G:

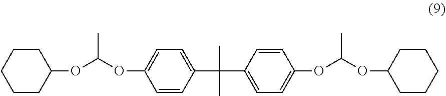 [SMILES: CC(Oc1ccc(C(C)(C)c2ccc(OC(C)OC3CCCCC3)cc2)cc1)OC1CCCCC1]

The results of the NMR analyses of Compound G are as follows.

¹H-NMR (400 MHz, acetone-d6): δ ppm=1.18 to 1.50 (12H), 1.39 (6H), 1.61 (6H), 1.65 (4H),1.81 (4H), 3.64 (2H), 5.49 (2H), 6.89 (4H), 7.12 (4H)

¹³C-NMR (400 MHz, acetone-d6): δ ppm=21.47, 24.48, 24.65, 26,31, 31.34, 32.96, 34.17, 42.26, 74.76, 98.95, 117.67, 128.29, 144.63, 155.74

### [Synthesis of Resist Polymer]

### Synthesis Example 1

### Synthesis of P-hydroxystyrene/Styrene/t-Butyl Methacrylate Copolymer

Into an eggplant flask equipped with a thermometer, a cooling tube, and a stirrer, 900.0 g of a p-ethylphenol solution (hereinafter, referred to as “PHS monomer solution”) containing 24% by mass of p-hydroxystyrene, 23% by mass of methanol and 10% by mass of water, was introduced, and heated to 80° C. Into another container, 1,350.0 g of a PHS monomer solution having the same composition as that described above, 184.6 g of styrene, 182.2 g of t-butyl acrylate, and 39.1 g of dimethyl 2,2′-azobis(2-methylpropionate) were introduced and stirred, to prepare a homogeneous solution. The resulting solution was added dropwise into the above-described eggplant flask over 2 hours, and stirring was continued for another hour to carry out the polymerization reaction. Thereafter, the resulting polymerization solution was cooled to room temperature, and the cooled polymerization solution was added dropwise into a mixed solution of 4,023 g of methylcyclohexane and 603 g of 2-propanol to allow the polymer to precipitate, and the supernatant was removed. Further, for the purpose of purification, the operation of adding 2-propanol to the polymer to re-dissolve the polymer, adding the resulting solution dropwise into methylcyclohexane to allow the polymer to precipitate, and removing the supernatant, was repeated five times.

The recovered polymer was dissolved in 2,600 g of PGMEA and concentrated under reduced pressure at 40° C., to prepare a PGMEA solution having a polymer concentration of 50% by mass. The resulting polymer had a composition ratio of p-hydroxystyrene: styrene: t-butyl methacrylate of 59.1:20.6:20.3, an Mw of 19,000 and a ratio Mw/Mn of 1.83.

### [Preparation of Resist Composition]

A quantity of 60 parts by mass (30 parts by mass in terms of Compound A) of the PGMEA solution containing 50% by mass of Compound A obtained in Example 1, 140 parts by mass (70 parts by mass in terms of the polymer) of the solution of the resist polymer obtained in Synthesis Example 1, 1 part by mass of an acid generator (SP140, manufactured by ADEKA Corporation) and 0.1 parts by mass of a surfactant (F447, manufactured by DIC Corporation) were dissolved in PGMEA. After adjusting the solid concentration to 40% by mass, the resulting solution was filtered with a membrane filter having a pore diameter of 0.45 μm, to prepare a resist composition.

The respective resist compositions were prepared in the same manner as described above, incorporating Compounds B to G prepared in Examples 2 to 5 and Comparative Examples 1 and 2, respectively, at the proportions shown in Table 1.

Further, a resist composition which does not contain any of the Compounds A to G was prepared, as the resist composition of Comparative Example 3.

Using each of the above-described resist compositions, the physical properties of each resist composition were evaluated, according to the following methods.

### [Crack Resistance and Surface Roughness]

Six-inch silicon wafers were each coated with hexamethyldisilazane, and subjected to a heat treatment at 100° C. for 60 seconds. Each silicon wafer after the treatment was spin-coated with each resist composition obtained above, and prebaked at 100° C. for 60 seconds on a hot plate, to form a resist layer having a film thickness of 8 μm on the wafer.

Each resulting resist layer was etched under the following conditions.

The evaluation of the crack resistance was carried out by visually observing the surface of each resist layer after etching, and the crack resistance was evaluated as “◯” when no crack was observed, and evaluated as “×” when cracks were observed. The evaluation results are shown in Table 1.

### <Etching Conditions>

Output: 180 W

Chamber pressure: 18 Pa

Gas type: O₂

Gas flow rate: 100 mL/min

Etching time: 10 minutes

Further, the determination of the surface roughness was carried out by measuring the surface of each resist layer after etching with an atomic force microscope (Dimension Icon, manufactured by Bruker Corporation), and calculating the average surface roughness Ra within an area of 5 μm square. The Ra is the arithmetic average roughness defined in JIS B 0601: 2013. Based on the Ra value of the resist layer formed from the resist composition (Comparative Example 3) which does not contain any of the Compounds A to G, the surface roughness of each resist layer was evaluated as “◯” when the Ra value was 60% or less, evaluated as “Δ” when the Ra value was more than 60% and less than 100%, and evaluated as “×” when the Ra value was 100% or more, with respect to the Ra value in Comparative Example 3. The evaluation results are shown in Table 1.

### [Evaluation of Sensitivity of Resist Composition]

Six-inch silicon wafers were each coated with hexamethyldisilazane, and subjected to a heat treatment at 100° C. for 60 seconds. Each silicon wafer after the treatment was spin-coated with each resist composition obtained above, and dried at 100° C. for 60 seconds on a hot plate, to form a resist layer having a film thickness of 8 μm on the wafer.

Using an open-flame exposure apparatus (UVS-2000, manufactured by Litho Tech Japan Corporation; light source: Hg-Xe lamp), each of the thus formed resist layers was subjected to 18 shots of 10 mm²×10 mm² irradiation with light having a wavelength of 248 nm, varying the amount of light exposure, and then subjected to a heat treatment at 100° C. for 60 seconds. Subsequently, each silicon wafer which had been exposed and heat-treated was developed with a 2.38% by mass aqueous solution of tetramethylammonium hydroxide under the condition of 23° C., using an apparatus for measuring resist development rate (RDA-790, manufactured by Litho Tech Japan Corporation), and the change over time in the resist film thickness at each light exposure amount was measured. The resulting data were analyzed, and the sensitivity Eth of each resist layer was determined. A smaller value of Eth indicates a higher sensitivity.

Eth: the amount of light exposure (mJ/cm²) corresponding to a residual film rate of 0%, when an approximate straight line is drawn within the range of residual film rate of from 10% to 70% in a residual film rate curve.

Based on the Eth value of the resist layer formed from the resist composition (Comparative Example 3) which does not contain any of the Compounds A to G, the sensitivity of each resist layer was evaluated as “◯” when the Eth value was 80% or less, and evaluated as “Δ” when the Eth value was more than 80%, with respect to the Eth value in Comparative Example 3. The evaluation results are shown in Table 1.

TABLE 1

| | | | | Acid | | | | |
|---|---|---|---|---|---|---|---|---|
| | Additive | | Polymer | generator | Surfactant | | | |
| | | [parts by | [parts by | [parts by | [parts by | Crack | Surface | |
| | Compound | mass] | mass] | mass] | mass] | Resistance | roughness | Sensitivity |
| Example 1 | A | 30 | 70 | 1 | 0.1 | ◯ | ◯ | ◯ |
| Example 2 | B | 10 | 90 | 1 | 0.1 | ◯ | ◯ | Δ |
| Example 3 | C | 30 | 70 | 1 | 0.1 | ◯ | ◯ | ◯ |
| Example 4 | D | 30 | 70 | 1 | 0.1 | ◯ | ◯ | ◯ |
| Example 5 | E | 30 | 70 | 1 | 0.1 | ◯ | ◯ | Δ |
| Comparative | F | 30 | 70 | 1 | 0.1 | ◯ | Δ | Δ |
| Example 1 | | | | | | | | |
| Comparative | G | 30 | 70 | 1 | 0.1 | ◯ | X | ◯ |
| Example 2 | | | | | | | | |
| Comparative | None | 0 | 70 | 1 | 0.1 | X | X | Δ |
| Example 3 | | | | | | | | |

Since Compound E in Example 5 contains a naphthalene ring, the absorption of light having a wavelength of 248 nm was strong, resulting in a sensitivity lower than that in Example 2. However, it is assumed that it is possible to reduce the occurrence of cracks and irregularities on the surface of the resulting resist film while maintaining the sensitivity, if the exposure is carried out with the i-ray (365 nm), EUV or the like.

When the resist composition containing the acetal compound according to the present invention was used for forming a thick resist film, it was possible to reduce the occurrence of cracks in the resist film after etching, and also to reduce the irregularities on the surface of the resist film which occur due to the formation of air bubbles.

### INDUSTRIAL APPLICABILITY

The acetal compound according to the present invention can be used in a resist composition, particularly, in a resist composition for forming a thick resist film.

1. An acetal compound represented by the following general formula (1): 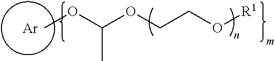
wherein, in the general formula (1), R¹ represents an alkyl group having from 1 to 12 carbon atoms, a cycloalkyl group having from 5 to 12 carbon atoms, or an acyl group having from 1 to 12 carbon atoms; n represents an integer from 1 to 5; m represents an integer from 2 to 4; and Ar represents a residue obtained by removing a hydrogen atom(s) from the hydroxyl groups of a polyhydric phenol having a valence of m.

2. The acetal compound according to claim 1, wherein the acetal compound is a reaction product of a polyhydric phenol having a valence of from 2 to 4 and a vinyl ether having an oxyethylene chain is represented by the following general formula (2): 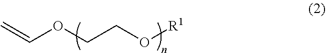
wherein, in the general formula (2), the definitions of R¹ and n are the same as those of R¹ and n in the general formula (1).

3. The acetal compound according to claim 1, wherein the polyhydric phenol is a phenol having from 10 to 30 carbon atoms.

4. The acetal compound according to claim 1, wherein the polyhydric phenol is a bisphenol type compound.

5. The acetal compound according to claim 1, wherein the polyhydric phenol is a naphthalenediol.

6. The acetal compound according to claim 1, wherein the polyhydric phenol is at least one compound selected from the group consisting of bisphenol A, 1,1,1-tris(4-hydroxyphenyl)ethane and 1,5-dihydroxynaphthalene.

7. The acetal compound according to claim 1, wherein the acetal compound is used in a resist composition.

8. An additive comprising the acetal compound according to claim 1, wherein the additive is used in a resist composition.

9. A resist composition comprising:
a polymer whose solubility in a developer changes by the action of an acid;
an acid generator;
a solvent; and
the acetal compound according to claim 1.

10. A method for producing the acetal compound according to claim 1,
wherein the method comprises allowing a polyhydric phenol having a valence of from 2 to 4 to react with a vinyl ether represented by the following general formula (2): 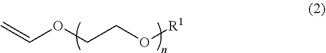
wherein, in the general formula (2), the definitions of R¹ and n are the same as those of R¹ and n in the general formula (1), in the presence of an acid.
